(19) United States
(12) Patent Application Publication
(10) Pub. No.: US 2021/0178079 A1
(43) Pub. Date: Jun. 17, 2021

(54) NOVEL COMPOSITE SAFETY SELF-DESTRUCTING SYRINGE

(71) Applicants: Xurong Huang, Huizhou City (CN); Haichuan Huang, Huizhou City (CN)

(72) Inventors: Xurong Huang, Huizhou City (CN); Haichuan Huang, Huizhou City (CN)

(21) Appl. No.: 16/745,344

(22) Filed: Jan. 17, 2020

(30) Foreign Application Priority Data
Dec. 16, 2019 (CN) 201922253594.8

(51) Int. Cl. A61M 5/315

(52) U.S. Cl. CPC A61M 5/31535; A61M 5/31513; A61M 5/3221; A61M 5/31591; A61M 5/31501; A61M 5/31576

(57) ABSTRACT

The invention discloses a novel composite safety self-destructing syringe, comprising cylinder, rod, ring, and needle hub; the cylinder comprises standard and widened wide sections; the inner wall width of the widened section is larger; the widened section is disposed on the top of the cylinder, and the standard section has a fixing groove; the rod is movably connected in the cylinder, and the bottom thereof is mated with the inner wall of the cylinder; the rod inside has a cavity, and a spring sleeve is disposed at the lower of the cavity. The fixing groove can mate with the convex reinforcement to achieve a good fixing effect. The rod has two different widths, making the sealing ring and the sealing rubber difficult to be rebounded. The exhaust hole is for labor-saving and facilitating retracting the syringe needle. The engagement block can better ensure the stability of the spring sleeve.

### BACKGROUND OF THE INVENTION

### 1. Field of the Invention

The invention relates to the technical field of syringes, in particular to a novel composite safety self-destructing syringe.

### 2. Description of the Related Art

With the widespread use of syringes, in order to avoid cross-infection of patients, more and more self-destructing syringes have appeared on the market. The self-destructing syringe is a disposable syringe product that can be prevented from being reused after use; after the injection is completed, the push rod cannot be pulled out of the syringe cylinder relying on manual or mechanical force to achieve self-locking, or the sealing performance of the piston is damaged, or the push rod of the syringe can be pushed to be broken, and finally the purpose of preventing reuse is achieved. At present, there are also many retractable self-destructing syringes on the market. This kind of syringe is provided with a specific assembly structure so that the syringe needle can be retracted into the syringe after the injection is completed, thereby achieving the self-destructing function. However, the safety performance of this type of retractable self-destructing syringe restricts the widespread promotion of the product. When users use a syringe, they usually need to draw the liquid medicine multiple times for dispensing. Since the air in the syringe needs to be completely exhausted, the syringe rod must be pushed to the zero position of the syringe, and this operation is prone to safety problems; when the syringe rod is pushed to the zero position, the self-destructing function of the syringe is triggered, causing the syringe to be unusable; on the other hand, when users use this type of syringe to inject drugs to patients, it is also easy to cause mistakes: during the injection process, the self-destructing function of the syringe is triggered, causing the needle tubing to be accidentally pulled out directly in the patient, which may lead to a medical accident.

### SUMMARY OF THE INVENTION

In order to solve the technical issues above, the invention provides the technical solutions as follows: a novel composite safety self-destructing syringe, comprising a syringe cylinder, a syringe rod, a sealing ring, and a needle hub, wherein the syringe cylinder comprises a standard wide section and a widened wide section, and the width of the inner wall of the widened wide section is larger than that of inner wall of the standard wide section; the widened wide section is disposed on the top of the syringe cylinder, and the standard wide section is provided with a fixing groove for fixing close to the widened wide section; the syringe rod is movably connected in the syringe cylinder, and the bottom of the syringe rod is tightly mated with the inner wall of the syringe cylinder; the inside of the syringe rod is provided with a cavity, and a spring sleeve that can move upward relative to the syringe rod is disposed at the lower part of the cavity; a compression spring is provided between the inside of the spring sleeve and the syringe rod; the outer wall of the syringe rod is provided with an exhaust hole; the top of the spring sleeve is provided with an engagement joint; a rubber sealing plug is engaged on the top of the spring rod; the inner wall of the spring rod is provided with an engagement block for abutting the bottom of the spring sleeve; the sealing ring is disposed on the bottom of the syringe cylinder; the middle of the sealing ring is provided with a through hole, and two sides of the sealing ring are provided with convex reinforcements that mate with the fixing groove; the needle hub comprises a first step face, a second step face, and a third step face, which are sequentially reduced in width from bottom to top; the needle hub is fixed in the through hole of the sealing ring through the first step face; the first step face is provided with an engagement groove that mates with the engagement joint, and the inside of the third step face is provided with a syringe needle.

Preferably, the engagement block has a quartering structure and is arranged in a ring shape on the inner wall of the syringe rod.

Preferably, the bottom of the sealing ring is provided with a concave surface that mates with the top of the rubber sealing plug.

Preferably, a rear cover is engaged on the top of the syringe rod.

Preferably, the inside of the rubber sealing plug is provided with a latching groove for latching the top of the syringe rod.

Preferably, a buffer cavity is formed between the first step face and the sealing ring.

Compared with the prior art, the invention has the following advantageous effects: a fixing groove is provided to mate with the convex reinforcement of the sealing ring to achieve a good fixing effect. The syringe rod adopts a design with two different widths, which can make the sealing ring and the sealing rubber difficult to be rebounded. The exhaust hole is for labor-saving and facilitating retracting the syringe needle. The engagement block is provided to better ensure the stability of the spring sleeve. The compression spring is hermetically closed in the middle, which can reduce winding, thereby reducing the assembly workload; at the same time, the syringe is provided with a pneumatic and spring-type retraction structure to ensure the safety of the syringe during use. A buffer cavity is provided in the syringe; when the syringe is used for dispensing, the self-destructing function is not triggered when the syringe rod is pushed to the zero position, so that the syringe rod can be repeatedly pushed to the zero position for safe dispensing.

### BRIEF DESCRIPTION OF THE DRAWINGS

FIG. 1 is a schematic view illustrating the overall structure of the novel composite safety self-destructing syringe of the invention.

FIG. 2 is a schematic view illustrating the structure of the syringe rod of the novel composite safety self-destructing syringe of the invention.

FIG. 3 is a schematic view illustrating the structure of the sealing ring of the novel composite safety self-destructing syringe of the invention.

FIG. 4 is a schematic view illustrating the structure of the needle hub of the novel composite safety self-destructing syringe of the invention.

FIG. 5 is a schematic view illustrating the structure of the rubber sealing plug of the novel composite safety self-destructing syringe of the invention.

FIG. 6 is a cross-sectional view illustrating the part of A-A in FIG. 2.

FIG. 7 is a schematic view illustrating the invention at the zero position state.

FIG. 8 is a schematic view illustrating the invention of the startup state.

FIG. 9 is a schematic view illustrating the invention of an initial self-destructing state.

FIG. 10 is a schematic view illustrating the invention of a complete self-destructing state.

FIG. 11 is a schematic view illustrating the structure of the compression spring of the novel composite safety self-destructing syringe of the invention.

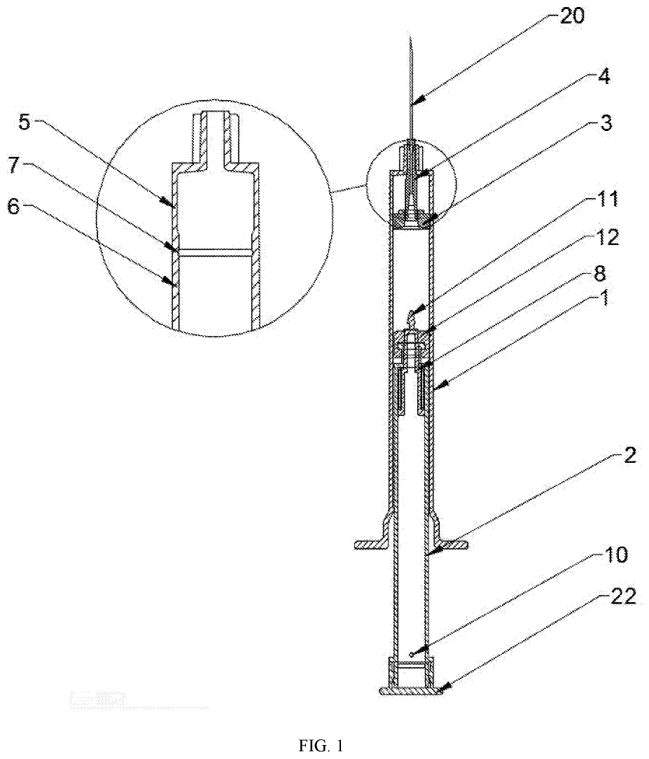

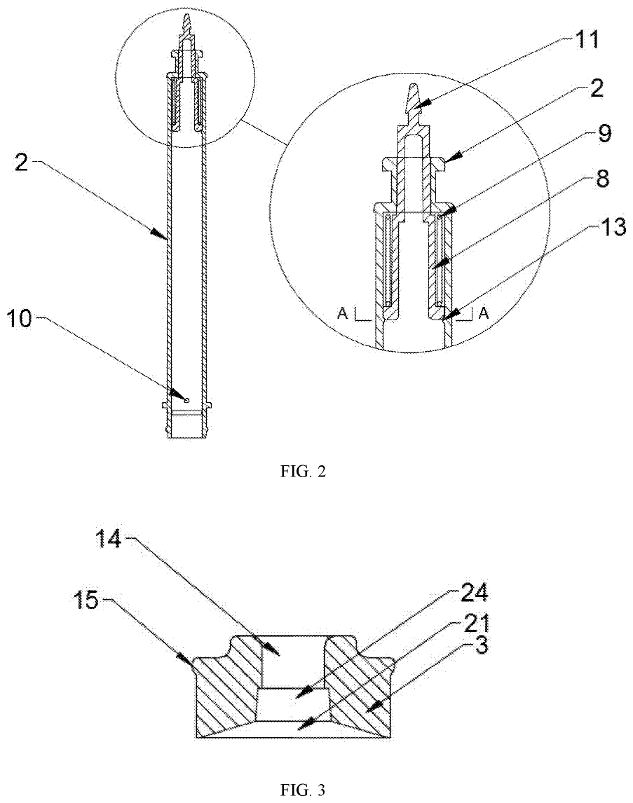

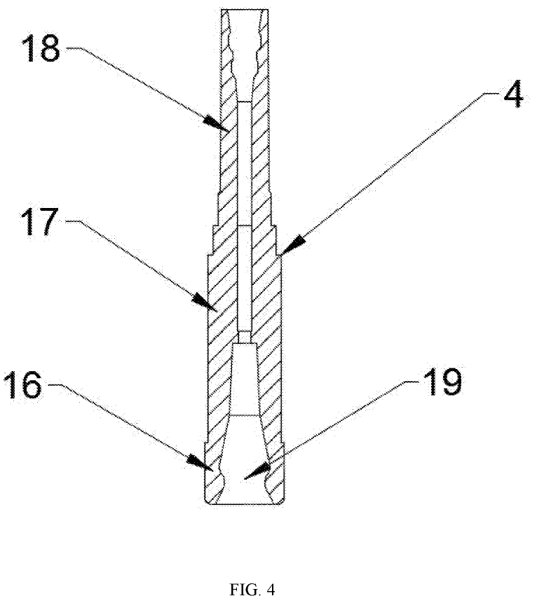

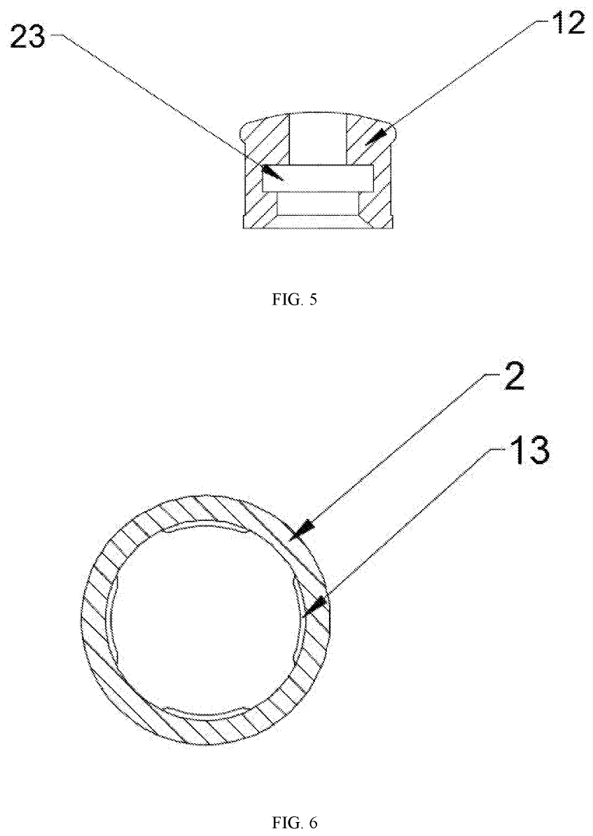

In the figures, 1 refers to the syringe cylinder; 2 refers to the syringe rod; 3 refers to the sealing ring; 4 refers to the needle hub; 5 refers to the standard wide section; 6 refers to the widened wide section; 7 refers to the fixing groove; 8 refers to the spring sleeve; 9 refers to the compression spring; 10 refers to the exhaust hole; 11 refers to the engagement joint; 12 refers to the rubber sealing plug; 13 refers to the engagement block; 14 refers to the through hole; 15 refers to the convex reinforcement; 16 refers to the first step face; 17 refers to the second step face; 18 refers to the third step face; 19 refers to the engagement groove; 20 refers to the syringe needle; 21 refers to the concave surface; 22 refers to the rear cover; 23 refers to the latching groove; 24 refers to the buffer cavity.

### DETAILED DESCRIPTION OF THE PREFERRED EMBODIMENTS

The invention is further described in detail with reference to the drawings hereinafter.

With reference to the drawings, a novel composite safety self-destructing syringe, comprising a syringe cylinder 1, a syringe rod 2, a sealing ring 3, and a needle hub 4, wherein the syringe cylinder 1 comprises a standard wide section 5 and a widened wide section 6, and the width of the inner wall of the widened wide section 6 is larger than that of inner wall of the standard wide section 5; the widened wide section 6 is disposed on the top of the syringe cylinder 1, and the standard wide section 5 is provided with a fixing groove 7 for fixing close to the widened wide section 6; the syringe rod 2 is movably connected in the syringe cylinder 1, and the bottom of the syringe rod 2 is tightly mated with the inner wall of the syringe cylinder 1; the inside of the syringe rod 2 is provided with a cavity, and a spring sleeve 8 that can move upward relative to the syringe rod 2 is disposed at the lower part of the cavity; a compression spring 9 is provided between the inside of the spring sleeve 8 and the syringe rod 2; the outer wall of the syringe rod 2 is provided with an exhaust hole 10; the top of the spring sleeve 8 is provided with an engagement joint 11; a rubber sealing plug 12 is engaged on the top of the spring rod 2; the inner wall of the spring rod 2 is provided with an engagement block 13 for abutting the bottom of the spring sleeve 8; the sealing ring 3 is disposed on the bottom of the syringe cylinder 1; the middle of the sealing ring 3 is provided with a through hole 14, and two sides of the sealing ring 3 are provided with convex reinforcements 15 that mate with the fixing groove 7; the needle hub 4 comprises a first step face 16, a second step face 17, and a third step face 18, which are sequentially reduced in width from bottom to top; the needle hub 4 is fixed in the through hole 14 of the sealing ring 3 through the first step face 16; the first step face 16 is provided with an engagement groove 19 that mates with the engagement joint 11, and the inside of the third step face 18 is provided with a syringe needle 20.

The engagement block 13 has a quartering structure and is arranged in a ring shape on the inner wall of the syringe rod 2.

The bottom of the sealing ring 3 is provided with a concave surface 21 that mates with the top of the rubber sealing plug 12.

A rear cover 22 is engaged on the top of the syringe rod 2.

The inside of the rubber sealing plug 12 is provided with a latching groove 23 for latching the top of the syringe rod 2.

A buffer cavity 24 is formed between the first step face 16 and the sealing ring 3.

In the invention, a design with two different widths of a widened wide section and a standard wide section is provided at the end of the syringe rod; the width of the inner wall of the widened wide section is larger than that of inner wall of the standard wide section, which can ensure that the sealing ring moves downward with the syringe rod more easily and effortlessly, and can also prevent the sealing ring and the rubber sealing plug from returning to the standard wide section. The end of the spring sleeve is engaged with the engagement block on the inner wall of the syringe rod to ensure the overall stability during use. The compression spring uses a design of tight connection in the middle, which can reduce the entanglement of the springs, and this design can reduce the workload whether it is automatic or manual assembly.

The method of using of the invention is as follows:

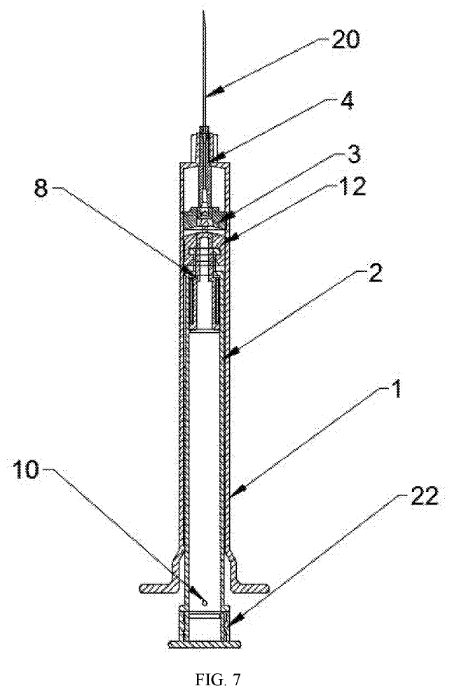

1. when it is necessary to use a syringe for dispensing, as shown in FIG. 7, the user pushes the syringe rod to the zero position; at this time, the engagement connector of the spring sleeve is located in the buffer cavity of the needle hub, and will not be engaged with the engagement groove on the needle hub, which avoids triggering the self-destructing function, and achieves safe dispensing;

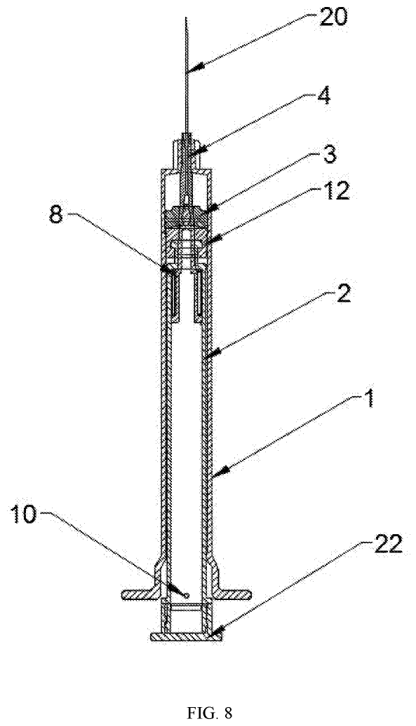

2. when the dispensing is completed, the drug needs to be injected into the patient, as shown in FIG. 8; the syringe rod is pushed to the activated state, and the engagement connector of the spring sleeve is located in the buffer cavity of the needle hub, and will not be engaged with the engagement groove on the needle hub, which does not trigger the self-destructing function, ensures safety during injection, and avoids medical accidents;

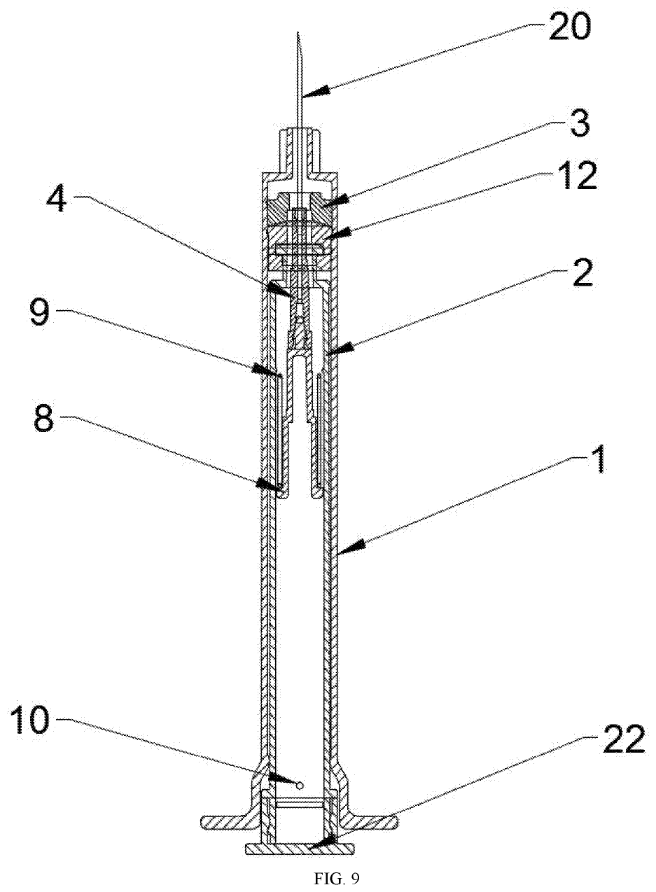

3. after the injection is completed and the syringe is pulled out of the human body, the syringe needs to be self-destructed, as shown in FIG. 9: pushing the syringe rod of the syringe down from the state shown in FIG. 8; the lower end of the syringe rod squeezes the sealing ring, so that the convex reinforcements on both sides of the sealing ring are released from the engagement of the fixing groove and move from the standard wide section to the widened wide section at the lower part; the elastic stress created by the compressed air generated by the gap between the sealing ring and the syringe cylinder on the first step face that pushes the needle hub upward is growing, until the engagement connector passes through the buffer cavity and is engaged with the engagement groove on the needle hub; continuing to push the syringe rod down to enable the end of the spring sleeve to be released from the engagement of the quartering engagement block; at the same time, since the width of the standard wide section is smaller than that of the widened wide section, and both sides of the sealing ring and the rubber sealing plug are provided with convex reinforcements, they will not return to the standard wide section when they enter into the widened wide section area;

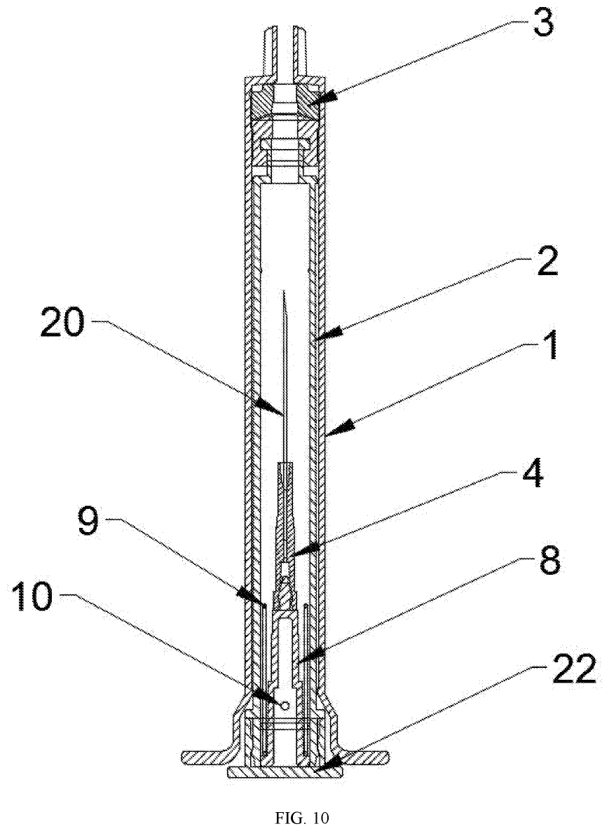

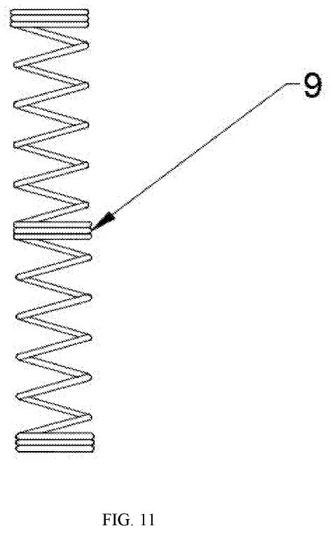

4. under the action of the compression spring, as shown in FIG. 10, the elastic stress of the spring sleeve and the elastic stress of the compressed air on the needle hub enable the spring sleeve and the needle hub to be sprung into the cavity of the syringe rod together; after the compression spring is stretched, it can press the spring sleeve at the top of the cavity of the syringe rod to ensure that the syringe needle does not fall back to be exposed out of the syringe rod; since the syringe rod is provided with an exhaust hole, it is more convenient for the syringe needle to be rebounded; with such a composite syringe assembly structure, users can clearly distinguish the steps when operating the syringe for injection and self-destructing, and each step provides clear operation feedback, which can ensure the safety of injection and enhance the reliability of self-destructing.

The invention and the embodiments thereof have been described above; the description is not restrictive; what is shown in the drawings is only one of the embodiments of the invention, and the actual structure is not limited thereto. Any structural manners and embodiments similar to the technical solution of the invention made by those of ordinary skill in the art who are inspired by the invention without creative efforts shall all fall within the protection scope of the invention.

1. A novel composite safety self-destructing syringe, comprising a syringe cylinder (1), a syringe rod (2), a sealing ring (3), and a needle hub (4), wherein the syringe cylinder (1) comprises a standard wide section (5) and a widened wide section (6), and the width of the inner wall of the widened wide section (6) is larger than that of inner wall of the standard wide section (5); the widened wide section (6) is disposed on the top of the syringe cylinder (1), and the standard wide section (5) is provided with a fixing groove (7) for fixing close to the widened wide section (6); the syringe rod (2) is movably connected in the syringe cylinder (1), and the bottom of the syringe rod (2) is tightly mated with the inner wall of the syringe cylinder (1); the inside of the syringe rod (2) is provided with a cavity, and a spring sleeve (8) that can move upward relative to the syringe rod (2) is disposed at the lower part of the cavity; a compression spring (9) is provided between the inside of the spring sleeve (8) and the syringe rod (2); the outer wall of the syringe rod (2) is provided with an exhaust hole (10); the top of the spring sleeve (8) is provided with an engagement joint (11); a rubber sealing plug (12) is engaged on the top of the spring rod (2); the inner wall of the spring rod (2) is provided with an engagement block (13) for abutting the bottom of the spring sleeve (8); the sealing ring (3) is disposed on the bottom of the syringe cylinder (1); the middle of the sealing ring (3) is provided with a through hole (14), and two sides of the sealing ring (3) are provided with convex reinforcements (15) that mate with the fixing groove (7); the needle hub (4) comprises a first step face (16), a second step face (17), and a third step face (18), which are sequentially reduced in width from bottom to top; the needle hub (4) is fixed in the through hole (14) of the sealing ring (3) through the first step face (16); the first step face (16) is provided with an engagement groove (19) that mates with the engagement joint (11), and the inside of the third step face (18) is provided with a syringe needle (20).

2. The novel composite safety self-destructing syringe according to claim 1, wherein the engagement block (13) has a quartering structure and is arranged in a ring shape on the inner wall of the syringe rod (2).

3. The novel composite safety self-destructing syringe according to claim 1, wherein the bottom of the sealing ring (3) is provided with a concave surface (21) that mates with the top of the rubber sealing plug (12).

4. The novel composite safety self-destructing syringe according to claim 1, wherein a rear cover (22) is engaged on the top of the syringe rod (2).

5. The novel composite safety self-destructing syringe according to claim 1, wherein the inside of the rubber sealing plug (12) is provided with a latching groove (23) for latching the top of the syringe rod (2).

6. The novel composite safety self-destructing syringe according to claim 1, wherein a buffer cavity (24) is formed between the first step face (16) and the sealing ring (3).
